(19) United States
(12) Patent Application Publication
(10) Pub. No.: US 2007/0021376 A1
(43) Pub. Date: Jan. 25, 2007

(54) Supplement composition and method of use in enhancement of methylation process

(75) Inventor: Vincent C. Giampapa, Little Falls, NJ (US)

Correspondence Address: MELVIN K. SILVERMAN, 500 WEST CYPRESS CREEK ROAD, SUITE 500, FT. LAUDERDALE, FL, 33309, US

(73) Assignee: Suracell, Inc., Montclair, NJ (US)

(21) Appl. No.: 11/483,688

(22) Filed: Jul. 10, 2006

Related U.S. Application Data
(60) Provisional application No. 60/702,057, filed on Jul. 21, 2005.

(51) Int. Cl. A61K 31/714; A61K 31/4415; A61K 31/525; A61K 31/205; A61K 31/10

(52) U.S. Cl. 514/52; 514/251; 514/350; 514/554; 514/708

(57) ABSTRACT

A supplement composition for enhancement of methylation process is provided, which contains vitamin B₆ (as pyridoxine HCl), folic acid, vitamin B₁₂ (as cyanocobalamin), betaine HCl, and methylsulfonylmethane; and also contains S-adenosylmethionine. The supplement composition further includes silymarin (from milk thistle seed extract), N-acetyl L-cysteine, and cruciferious blend which includes broccoli (brassica oleracea var. talica), kale (brassica oleracea var. acephala), and radish (raphanus sativus). Further provided is a method of using the supplement composition for enhancement of methylation process.

### CROSS REFERENCE TO RELATED APPLICATION

This application claims the benefit under 35 USC 119 (e) of the provisional patent application Ser. No. 60/702,057, filed Jul. 21, 2005, which is herein incorporated by reference in its entirety.

### FIELD OF THE INVENTION

The present invention relates to dietary supplement compositions and the method of use thereof for enhancement of an individual's methylation process.

### BACKGROUND OF THE INVENTION

DNA methylation is the covalent addition of a methyl group to the 5-carbon position of cytosine, predominantly within cytosine guanine dinucleotides (CpG) (Christman, J. K., et al, Reversibility of changes in nucleic acid methylation and gene expression induced in rat liver by severe dietary methyl deficiency, Carcinogenesis 14:551-557, 1993). In the “normal” situation, 60-90% of all CpG sequences in the genome are methylated, whereas unmethylated CpG dinucleotides are mainly clustered in CpG-rich sequences, termed “CpG islands”, within the promoter regions of genes (Ng, H. H. & Bird, A., DNA methylation and chromatin modification. Curr. Opin. Genet. Dev. 9:158-163, 1999). Normally, both core promoter and transcription start site are included within the CpG island, and if the corresponding gene transcription factors are available and the CpG island remains in an unmethylated state with an open chromatin configuration associated with hyperacetylated histones, the transcription of that particular gene will occur (Esteller, M. et al, Cancer as an epigenetic disease: DNA methylation and chromatin alterations in human tumors. J. Pathol. 196:1-7, 2002). Methylation of CpG islands, however, inhibits gene transcription by directly impeding the binding of transcription factors to their cis-acting sites and/or by promoting the binding of methyl-DNA-binding proteins, which restrict access of transcription factors to DNA (Baylin, S. B., et al, Alterations in DNA methylation: a fundamental aspect of neoplasia. Adv. Cancer Res. 72:141-196, 1998). Thus, DNA methylation appears to be an important epigenetic mechanism of transcriptional control.

Furthermore, in mammals methylation has also been proposed to be a genome defense system against foreign DNA such as viruses. Viruses that infect cells and integrate into the host cell DNA frequently become methylated. Methylation can inactivate a promoter and thereby silence gene expression from a viral promoter. In addition to silencing gene expression from foreign DNA promoters, methylation has also been shown to prevent DNA sequences such as transposons from moving to a new site in the DNA. In this way, methylation can limit the spread of infectious virus from cell to cell, and prevent the damaging spread of transposon sequences.

Recent studies have demonstrated that methyl insufficiency and/or abnormal DNA methylation likely have significant roles in the development of several pathologies including birth defects, cancer, diabetes, heart disease and neurological disorders (Costello, J. F. et al, Methylation matters, J. Med. Genet 38:285-303, 2001; and Poirier, L. A., et al, Blood S-adenosylmethionine concentrations and lymphocyte methylenetetrahydrofolate reductase activity in diabetes mellitus and diabetic nephropathy, Metabolism 50:1014-1018, 2001). The evidence that DNA methylation is influenced by diet and dietary factors arises from both preclinical and clinical studies. For example, liver DNA is hypomethylated in rats continuously fed methyl-deficient diets (Christman, J. K., et al, Reversibility of changes in nucleic acid methylation and gene expression induced in rat liver by severe dietary methyl deficiency, Carcinogenesis 14:551-557, 1993). Hypomethylation of specific cytosine guanine dinucleotide (CpG) sites within several genes, for example, c-myc (myelocytomatosis oncogene), c-fos (FBJ osteosarcoma oncogene) and H-ras (Harvey rat sarcoma virus oncogene), also was observed in rat liver. Moreover, folate depletion resulted in lymphocyte DNA hypomethylation in postmenopausal women, which was reversed following folate repletion (Jacob, R. A., et al, Moderate folate depletion increases plasma homocysteine and decreases lymphocyte DNA methylation in postmenopausal women. J. Nutr. 128:1204-1212, 1998).

Dietary factors that are likely to have an impact on DNA methylation processes include folate, vitamin B₁₂ (cobalamin), vitamin B₆ (pyridoxine), vitamin B₂ (riboflavin), methionine, choline and alcohol. Normally in methyl metabolism, a carbon unit from serine or glycine is transferred to tetrahydrofolate (THF) to form 5,10-methylenetetrahydrofolate. This compound is either used for the synthesis of thymidine, which is incorporated into DNA, oxidized to formyl-THF for the synthesis of purines, which are building blocks of RNA and DNA, or reduced to 5-methyltetrahydrofolate and used to methylate homocysteine to form methionine. Methionine is converted to S-adenosylmethionine, a universal donor of methyl groups, which methylates DNA, RNA, hormones, neurotransmitters, membrane lipids, proteins and other molecules (Selhub, J., Folate, vitamin B12 and vitamin B6 and one carbon metabolism. J. Nutr. Health Aging 6:39-42, 2002).

Furthermore, vitamins B₆, B₁₂ and folic acid have been shown to work together to reduce blood levels of homocysteine, an amino acid normally found in the body that, in excess levels, has been linked to an increased risk of cardiovascular disease.

Betaine hydrochloride (betaine HCl) is considered as a non-essential nutrient and a source of hydrochloric acid, a naturally occurring chemical in the stomach that helps digest food by breaking up fats and proteins. Betaine HCl aids the absorption of folic acid, vitamin C, beta-carotene, iron, calcium, magnesium and zinc. Numerous studies have shown stomach acid secretion declines with advancing age. Such decline may increase the risk of malabsorption and certain clinical conditions such as osteoporosis, asthma, eczema, chronic hives, psoriasis, arthritis, thyroid disorder, vitiligo, chronic candida, GI infections and parasites. Betaine HCl is naturally produced by the parietal cells of the stomach, no food source exists for betaine HCl.

Methylsulfonylmethane, abbreviated MSM, is an organic sulfur-containing compound that occurs naturally in a variety of fruits, vegetables, grains and in animals, including humans in at least trace amounts. MSM is a metabolite of dimethyl sulfoxide. MSM has been used for pain relief, particularly in arthritis, immune modulation in autoimmune disorders, muscle repair, sleep aid and diabetes therapy.

N-acetyl L-cysteine is an amino acid and antioxidant. N-acetyl L-cysteine has been shown to play a protective role against a variety of toxic hazards such as cigarette smoke, auto exhaust, certain herbicides, and overdoses of acetaminophen. It has also been shown to have antiviral effects in patients with HIV due to inhibition of viral stimulation by reactive oxygen intermediates.

S-Adenosylmethionine (SAMe) is a naturally occurring compound that is involved in many biochemical processes in the body. SAMe is not found in food; it is produced by the body from ATP and the amino acid methionine. Deficiencies in methionine, folate, or vitamin B₁₂ can reduce SAMe levels. SAMe plays a role in the immune system, maintains cell membranes, and helps to produce and break down brain chemicals such as serotonin, melatonin, and dopamine as well as vitamin B12. SAMe also participates in the making of genetic materials DNA and cartilage. Numerous scientific studies indicate that SAMe may be useful in the treatment of depression, osteoarthritis, fibromyalgia, and liver disorders.

Silymarin refers to a mixture of three biochemicals isolated from milk thistle seed, which are silychristine, silydianin, and silybin. Silymarin has been traditionally used in the treatment of liver disease, and other conditions.

Broccoli is well known for its vitamin-rich, high in fiber, and low in calorie properties. Broccoli contains antioxidants including vitamin C and beta carotene. It is also rich in phytochemicals. Indole carbinol and sulforaphane are two different phytochemicals that are found in broccoli. It has been reported that beta carotene lowers the risk of heart attacks. A study done by researchers at Harvard Medical School report that men with clogged arteries who were fed beta carotene supplements suffered half as many heart seizures and strokes as did men given placebo pills.

Kale (Brassica oleracea var. acephala) is a hardy cabbage with coarse curly leaves that do not form a head, and an extremely nutritious food. It contains numerous phytochemicals, and is particularly rich in vitamin C and calcium.

Radish (Raphanus sativus) belongs to the mustard family, along with cabbage, cauliflower, kale and turnips. Radish is rich in potassium, vitamin C, folate, and magnesium. It is diuretic, and stimulates the appetite and digestion. Radish has also been found to have antipyretic, antibiotic and antihypertensive properties and has been used as cholesterol reducing aid and mild laxative.

The above-described nutrients have been used individually, or in a partial combination in nutritional supplements. It is desirable to provide a dietary supplement composition including the above-described active components, which function synergetically to provide further enhancement of an individual's methylation process.

### SUMMARY OF THE INVENTION

In one aspect, the present invention is directed to a supplement composition for enhancement of methylation process. In one embodiment, the supplement composition comprises effective amounts of vitamin B₆ (as pyridoxine HCl), folic acid, vitamin B₁₂ (as cyanocobalamin), betaine HCl, and methylsulfonylmethane. In a further embodiment, the supplement composition further comprises effective amounts of silymarin (from milk thistle seed extract), N-acetyl L-cysteine, and cruciferious blend which includes broccoli (brassica oleracea var. talica), kale (brassica oleracea var. acephala), and radish (raphanus sativus). Additionally, the supplement composition further comprises an effective amount of S-adenosylmethionine. Moreover, the supplement composition also comprises pharmaceutically acceptable excipients, and can be provided in a form for oral administration.

In a further aspect, the present invention is directed to a method for enhancement of an individual's methylation process and inhibition of DNA insertion or deletion by transposon. In one embodiment, as a regular strength treatment, the method comprises orally administering a supplement composition to a person daily in a dosage comprising from about 75 mg to about 400 mg of vitamin B₆ (as pyridoxine HCl), from about 35 μg to 200 μg of folic acid, from about 125 μg to 700 μg of vitamin B₁₂ (as cyanocobalamin), from about 125 mg to 700 mg of betaine HCl, and from about 100 mg to 600 mg of methylsulfonylmethane. Additionally, the supplement composition further includes about 200 mg to about 1200 mg of S-adenosylmethionine.

In another embodiment, as an extra strength treatment, the method comprises orally administering a supplement composition to a person daily in a dosage comprising from about 75 mg to about 400 mg of vitamin B₆ (as pyridoxine HCl), from about 35 μg to 200 μg of folic acid, from about 125 μg to 700 μg of vitamin B₁₂ (as cyanocobalamin), from about 125 mg to 700 mg of betaine HCl, from about 100 mg to 600 mg of methylsulfonylmethane, from about 75 mg to about 300 mg of silymarin (from milk thistle seed extract), from about 35 mg to about 150 mg of N-acetyl L-cysteine, and from about 75 mg to about 300 mg of cruciferious blend comprising broccoli (brassica oleracea var. italica), kale (brassica oleracea var. acephala), and radish (raphanus sativus). Additionally, the supplement composition further includes about 200 mg to about 1200 mg of S-adenosylmethionine.

### DETAILED DESCRIPTION OF THE INVENTION

In one embodiment, the present invention provides a dietary supplement composition for enhancing methylation process of a person in need thereof. The dietary supplement composition comprises vitamin B₆, folic acid, vitamin B₁₂, betaine HCl, methylsulfonylmethane (MSM), and pharmaceutically acceptable excipients.

Vitamin B₆ (as pyridoxine HCl) is included in the supplement compositions described herein. The active forms of pyridoxine, pyridoxal-5′-phosphate (PLP) and pyridoxamine-5′-phosphate, are coenzymes for numerous enzymes and as such, are essential for gluconeogenesis, niacin formation, and erythrocyte metabolism. Pyridoxine is a coenzyme for both cystathionine synthase and cystathionase, enzymes that catalyze the formation of cysteine from methionine. Homocysteine is an intermediate in this process and elevated levels of plasma homocysteine are recognized as a risk factor for vascular disease. The administration of pyridoxine has been reported to reduce the levels of homocysteine. In one embodiment, the supplement composition comprises pyridoxine in the form of pyridoxine HCl, preferably in the amount from about 75 mg to about 400 mg in one dosage. In a preferred embodiment, the supplement composition comprises about 150 mg of vitamin B6 in the form of pyridoxine HCl in one dosage. In another preferred embodiment, the supplement composition comprises about 200 mg of vitamin B₆ in the form of pyridoxine HCl in one dosage.

Herein, one dosage is also referred to as one serving. If the supplement composition is provided in the form of tablet, one dosage can be either one tablet, or two to three tablets. For example, 150 mg of vitamin B₆ in the form of pyridoxine HCl in one dosage can be provided in one tablet as shown in Supplement Composition A of Example 1; and 200 mg of vitamin B₆ in the form of pyridoxine HCl in one dosage can be provided in two tablets as shown in Supplement Composition B of Example 2. The size and number of tablets may depend on the manufacturability, which may further depend on the properties of the components and the pharmaceutically acceptable excipients used.

Folic acid, also called folate or methylfolate, is essential for the formation of red and white blood cells within bone marrow and also plays a role in heme formation. Folic acid in its active form, tetrahydrofolate, is a coenzyme that is involved in the transfer of methyl groups and it plays a role in DNA synthesis, purine synthesis, and amino acid synthesis, such as the conversion of glycine to serine and the transformation of homocysteine tc methionine. The activation of folic acid requires a vitamin B₁₂-dependent transmethylation and vitamin B₁₂ is also necessary for folic acid delivery to tissues. In one embodiment, the supplement composition comprises folate in the form of folic acid, preferably in the amount rom about 35 μg to about 200 μg in one dosage. In one preferred embodiment, the supplement composition comprises about 75 μg of folic acid in one dosage. In another preferred embodiment, the supplement composition comprises about 100 μg of folic acid in one dosage.

Vitamin B₁₂ (cobalamin) is another important vitamin included in the supplement compositions described herein. It is known that cobalamin can be converted to the active coenzymes, methylcobalamin and 5′-deoxyadenosylcobalamin. These coenzymes are necessary for folic acid metabolism, conversion of coenzyme A, and myelin synthesis. For example, methylcobalamin catalyzes the demethylation of a folate cofactor, which is involved in DNA synthesis. A lack of demethylation may result in folic acid deficiency. Deoxyadenosylcobalamin is the coenzyme for the conversion of methylmalonyl-CoA to succinyl-CoA, which plays a role in the citric acid cycle. Importantly, cobalamin, along with pyridoxine and folic acid is implicated in the proper metabolism of homocysteine. Cobalamin is available as cyanocobalamin, methylcobalamin, hydroxocobalamin, adenosylcobalamin, and hydroxycyanocobalamin. In one embodiment, the supplement composition comprises vitamin B₁₂ in the form of cyanocobalamin, preferably in the amount from about 125 μg to about 700 μg. In one preferred embodiment, the supplement composition comprises about 250 μg of cyanocobalamin in one dosage. In another preferred embodiment, the supplement composition comprises about 350 μg of cyanocobalamin in one dosage.

Betaine HCl is naturally produced by the parietal cells of the stomach, no food source exists for betaine HCl. Numerous studies have shown stomach acid secretion declines with advancing age. Such decline may increase the risk of malabsorption and certain clinical conditions. In one embodiment, the supplement composition comprises betaine HCl, preferably in the amount from about 125 mg to about 700 mg. In a preferred embodiment, the supplement composition comprises about 250 mg of betaine HCl in one dosage. In another preferred embodiment, the supplement composition comprises about 350 mg of betaine HCl in one dosage.

Methylsulfonylmethane (MSM) is a water-soluble, solid compound and is available commercially from various sources. In one embodiment, the supplement composition comprises MSM, preferably in the amount from about 100 mg to about 600 mg in one dosage. In a preferred embodiment, the supplement composition comprises about 200 mg of MSM in one dosage. In another preferred embodiment, the supplement composition comprises about 300 mg of MSM in one dosage.

Example 1 provides an exemplary supplement composition of the present invention, which is considered as a regular strength formula.

In a further embodiment, the dietary supplement composition of the present invention further comprises N-acetyl cysteine, silymarin (from milk thistle seed extract), and cruciferious blend which comprises broccoli (Brassica oleracea var. italica), kale (Brassica oleracea var. acephala), and radish (Raphanus sativus).

N-acetyl L-cysteine is an amino acid and antioxidant. N-acetyl L-cysteine is a precursor in the body to the critical antioxidant glutathione, which is produced within all cells, particularly by the liver. N-acetyl L-cysteine, as a compound that increases glutathione levels, protects cells against chrysotile toxicity. In one embodiment, the supplement composition comprises N-acetyl L-cysteine, preferably in the amount from about 35 mg to about 150 mg in one dosage.

Silymarin is a mixture of three biochemicals isolated from milk thistle seed, including silychristine, silydianin, and silybin. It is reported that silymarin may control cell membrane permeability, inhibit chemical pathways leading to inflammatory biochemicals, and increase protein production by liver cells, and it may have free radical scavenging properties. In one embodiment, the supplement composition comprises silymarin, preferably in the amount from about 35 mg to about 300 mg in one dosage. In a preferred embodiment, the supplement composition comprises about 150 mg of silymarin in one dosage.

Broccoli is rich in vitamin C, beta carotene, phytochemicals such as indole carbinol and sulforaphane and fiber. It has been reported that supplement containing beta carotene improves heart conditions, and lowers the risk of heart attacks. Kale (Brassica oleracea var. acephala) contains numerous phytochemicals, and is particularly rich in vitamin C and calcium. Radish is rich in potassium, vitamin C, folate, and magnesium. In one embodiment, the supplement composition comprises the cruciferious blend containing broccoli (Brassica oleracea var. italica), kale (Brassica oleracea var. acephala) and radish (Raphanus sativus), preferably in the amount from about 75 mg to about 300 mg in one dosage. In a preferred embodiment, the supplement composition comprises about 150 mg of the cruciferious blend described above in one dosage.

It has been found that the addition of silymarin, N-acetyl cysteine and cruciferious blend into the supplement composition described above provides further enhancement to the methylation process, which is particularly suitable for individuals who has deficiency in methylation process due to genetic predisposition, clinical conditions, advanced aging, or poor environmental impact, and therefore, particularly in need of enhancement of methylation process. In a preferred embodiment, the supplement composition comprises about 150 mg of silymarin, about 75 mg of N-acetyl cysteine, and about 150 mg of the cruciferious blend containing broccoli (Brassica oleracea var. italica), kale (Brassica oleracea var. acephala) and radish (Raphanus sativus) in one dosage.

Example 2 provides an exemplary supplement composition of the present invention, which is considered as an extra strength formula. As shown, the amounts of the active components in one dosage in the extra strength formula are higher than those in the regular formula. Furthermore, the additional active components described above provide further effectiveness in enhancing the methylation process.

In another embodiment, the supplement composition of the present invention further comprises S-adenosylmethionine. As a naturally occurring compound, S-Adenosylmethionine (SAMe) involves in many biochemical processes in the body and it is a universal donor of methyl groups, which methylates DNA and RNA. S-adenosylmethionine is available in the forms of S-adenosylmethionine butanedisulfonate, S-adenosylmethionine disulfate ditosylate, S-adenosylmethionine disulfate tosylate and S-adenosylmethionine tosylate. In one embodiment, the supplement composition comprises S-adenosylmethionine disulfate ditosylate, preferably in the amount from about 200 mg to about 1200 mg in one dosage. In one exemplary embodiment, Supplement Composition A of Example 1 further comprises 400 mg of S-adenosylmethionine (as S-adenosylmethionine disulfate ditosylate or other available forms) in one tablet. In another exemplary embodiment, Supplement Composition B of Example 2 further comprises 800 mg of S-adenosylmethionine (as S-adenosylmethionine disulfate ditosylate or other available forms) in per serving or two tablets.

As a convenient form of dietary supplement, the supplement compositions described above are provided in the form of tablet. However, it should be understood that tablet is only one of various convenient dosage forms which can be used for the supplement composition. Other suitable forms include hard or soft-gelatin capsules, powders, or in liquid dosage forms, such as elixirs, syrups, dispersed powders or granules, emulsions, or aqueous or oily suspensions. When other dosage forms are used, the amounts of the active components in one dosage remain the same; however, the concentration of the component in different pharmaceutical media can be different.

Preferably, the supplement composition is formulated as a tablet, and as such it can contain pharmaceutically acceptable excipients, according to methods and procedures well known in the art. As used herein, “excipients” means substances that are of little or no therapeutic value, but useful in the manufacture and compounding of various pharmaceutical preparations, which form the medium of the supplement composition. These substances include coloring, flavoring, and diluting agents; emulsifying and suspending agents; ointment bases; pharmaceutical solvents; antioxidants and preservatives for the product; and miscellaneous agents. Suitable excipients are described in Remington's Pharmaceutical Sciences, Mack Publishing Company, a standard reference text in this field, which is incorporated herein by reference in its entirety.

As used herein, “tablets” are solid pharmaceutical dosage forms containing active ingredients with or without suitable diluents and prepared either by compression or molding methods well known in the art. Although tablets are most frequently discoid in shape, they may also be round, oval, oblong, cylindrical, or triangular. They may differ greatly in size and weight depending on the amount of active ingredients present and the intended method of administration. They are divided into two general classes, (1) compressed tablets, and (2) molded tablets or tablet triturates. In addition to the active ingredients, tablets contain a number of inert excipients or additives. A first group of such excipients includes those materials that help to impart satisfactory compression characteristics to the formulation, including diluents, binders, and lubricants. A second group of such excipients helps to give additional desirable physical characteristics to the finished tablet, such as disintegrators, colors, flavors, and sweetening agents. Compressed tablets can be uncoated or can be sugar coated or film coated by known techniques to mask any unpleasant taste and protect the tablet from the atmosphere, or enteric coated for selective disintegration and adsorption in the gastrointestinal tract.

As used herein, “diluents” are inert substances added to increase the bulk of the formulation to make the tablet a practical size for compression. Commonly used diluents include calcium phosphate, calcium sulfate, lactose, kaolin, mannitol, sodium chloride, dry starch, powdered sugar, silica, and other suitable materials. As used herein, “binders” are agents used to impart cohesive qualities to the powdered material. Binders insure the tablet remaining intact after compression, as well as improving the free-flowing qualities by the formulation of granules of desired hardness and size. Materials commonly used as binders include starch; gelatin; sugars, such as sucrose, glucose, dextrose, molasses, and lactose; natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, and larch arabogalactan, and other suitable materials. As used herein, “lubricants” are materials that perform a number of functions in tablet manufacture, such as improving the rate of flow of the tablet granulation, preventing adhesion of the tablet material to the surface of the dies and punches, reducing interparticle friction, and facilitating the ejection of the tablets from the die cavity. Commonly used lubricants include talc, magnesium stearate, calcium stearate, stearic acid, and hydrogenated vegetable oils. As used herein, “coloring agents” are chemicals that give tablets a more pleasing appearance, and in addition help the manufacturer to control the product during its preparation and help the user to identify the product. Any of the approved certified water-soluble FD&C dyes, mixtures thereof, can be used to color tablets.

In a further aspect, the present invention provides the method of using the supplement compositions described above as a dietary supplement to enhance methylation process of an individual and inhibition of DNA insertion or deletion by transposon. Preferably, the supplement composition is administrated daily. The preferred daily dosage includes from about 75 mg to about 400 mg of vitamin B₆ (as pyridoxine HCl), from about 35 μg to about 200 μg of folate (as folic acid), from about 125 μg to about 700 μg of vitamin B₁₂ (as cyanocobalamin), from about 125 mg to about 700 mg of betaine HCl, from about 100 mg to about 600 mg of methylsulfonylmethane. Furthermore, the daily dosage further includes from about 75 mg to about 300 mg of silymarin (from milk thistle seed extract), from about 35 mg to about 150 mg of N-acetyl L-cysteine, and from about 75 mg to about 300 mg of the curciferous blend containing broccoli (Brassica oleracea var. italica), kale (Brassica oleracea var. acephala), radish (Raphanus sativus) daily.

Supplement Composition A of Example 1 is considered as a regular strength formula for enhancing methylation process. The preferred daily dosage is one tablet of Supplement Composition A, which includes about 150 mg of vitamin B₆ (pyridoxine HCl), about 75 μg of folate (as folic acid), about 250 μg of vitamin B₁₂ (as cyanocobalamin), about 250 mg of betaine HCl, and about 200 mg of methylsulfonylmethane. As described above, Supplement Composition A can further include about 400 mg of S-adenosylmethionine (as S-adenosylmethionine disulfate ditosylate or other available forms). Supplement Composition B of Example 2 is considered as an extra strength formula, which is particularly suitable for individuals who has deficiency in methylation process due to genetic predisposition, clinical conditions, advanced aging, or poor environmental impact, and therefore, particularly in need of enhancement of methylation process. For these individuals, a preferred daily dosage is two tablets of Supplement Composition B, which has a daily dosage of about 200 mg of vitamin B₆ (pyridoxine HCl), about 100 μg of folate (as folic acid), about 350 μg of vitamin B₁₂ (as cyanocobalamin), about 350 mg of betaine HCl, about 300 mg of methylsulfonylmethane, about 150 mg of silymarin (from milk thistle seed extract), about 75 mg of N-acetyl L-cysteine, and about 150 mg of the curciferous blend containing broccoli (Brassica oleracea var. italica), kale (Brassica oleracea var. acephala), radish (Raphanus sativus). As described above, Supplement Composition B can further include about 800 mg of S-adenosylmethionine (as S-adenosylmethionine disulfate ditosylate or other available forms).

A method of determining an individual's genetic predisposition for deficiency in methylation process is described in a co-pending patent application Ser. No. 60/796,423, entitled “Method of Determining Genetic Predisposition for Deficiency in Health Functions Using SNP Analysis”, which is herein incorporated by reference in its entirety. More specifically, upon performing a SNP genotyping assay of a biological sample collected from an individual, the individual's genetic predisposition for methylation process can be determined by using a specific methylation SNP panel which comprises predetermined methylation identifier SNPs. Such a SNP analysis determines and identifies an individual's genetic predisposition for methylation process as normal, sub-normal, and deficient. The individual whose genetic predisposition for methylation process is considered deficient is more likely to develop clinical conditions directly or indirectly related to the methylation process.

In a further aspect, the method of the present invention provides a suitable supplement composition based on an individual's genetic predisposition for methylation process. In one embodiment, an individual whose genetic predisposition for methylation process is sub-normal is recommended to take one tablet of Supplement Composition A of Example 1 daily. In another embodiment of the present invention, an individual whose genetic predisposition for methylation process is deficient is recommended to take two tablets of Supplement Composition B of Example 2 daily. By providing a suitable supplement composition based on an individual's genetic predisposition, the method of the present invention can more effectively assist in enhancing the methylation process and reducing the likelihood of the individual in developing clinical conditions caused by deficiency in methylation process.

The following examples are illustrative of the invention and are in no way to be interpreted as limiting the scope of the invention, as defined in the claims.

### EXAMPLE 1

A supplement composition of the following formulation was prepared in tablet form, including the appropriate excipients, by standard methods known to those of ordinary skill in the art:

TABLE 1
Supplement Composition A

| | Amount Per Serving |
|---|---|
| Contents | (in one tablet) |
| Vitamin B₆ (as pyridoxine HCl) | 150 mg |
| Folate (as folic acid) | 75 μg |
| Vitamin B₁₂ (as cyanocobalamin) | 250 μg |
| Betaine HCl | 250 mg |
| MSM | 200 mg |

Other ingredients include: dicalcium phosphate, microcrystalline cellulose, croscarmellose sodium, stearic acid, magnesium stearate, silica, and pharmaceutical glaze. In this example, each tablet has a weight from about 1100 mg to about 1400 mg.

### EXAMPLE 2

A supplement composition of the following formulation was prepared in tablet form, including the appropriate excipients, by standard methods known to those of ordinary skill in the art:

TABLE 2
Supplement Composition B

| | Amount Per Serving |
|---|---|
| Contents | (in two tablets) |
| Vitamin B₆ (as pyridoxine HCl) | 200 mg |
| Folate (as folic acid) | 100 μg |
| Vitamin B₁₂ (as cyanocobalamin) | 350 μg |
| Betaine HCl | 350 mg |
| MSM | 300 mg |
| Silymarin (from milk thistle seed extract) | 150 mg |
| N-acetyl L-cysteine | 75 mg |
| Cruciferious Blend: | 150 mg |
| Broccoli (Brassica oleracea var. italica), | |
| Kale (Brassica oleracea var. acephala), | |
| Radish (Raphanus sativus) | |

Other ingredients include: dicalcium phosphate, microcrystalline cellulose, croscarmellose sodium, stearic acid, magnesium stearate, silica, and pharmaceutical glaze. In this example, each tablet has a weight from about 1100 mg to about 1400 mg.

While the present invention has been described in detail and pictorially shown in the accompanying drawings, these should not be construed as limitations on the scope of the present invention, but rather as an exemplification of preferred embodiments thereof. It will be apparent, however, that various modifications and changes can be made within the spirit and the scope of this invention as described in the above specification and defined in the appended claims and their legal equivalents.

What is claimed is:

1. A supplement composition for enhancement of methylation process, comprising effective amounts of:
(a) vitamin B₆ (as pyridoxine HCl);
(b) folic acid;
(c) vitamin B₁₂ (as cyanocobalamin);
(d) betaine HCl; and
(e) methylsulfonylmethane.

2. The supplement composition of claim 1, wherein said composition comprises from about 75 mg to about 400 mg of said vitamin B₆ (as pyridoxine HCl) in one dosage.

3. The supplement composition of claim 1, wherein said composition comprises from about 35 μg to about 200 μg of said folic acid in one dosage.

4. The supplement composition of claim 1, wherein said composition comprises from about 125 μg to about 700 μg of said vitamin B₁₂ (as cyanocobalamin) in one dosage.

5. The supplement composition of claim 1, wherein said composition comprises from about 125 mg to about 700 mg of said betaine HCl in one dosage.

6. The supplement composition of claim 1, wherein said composition comprises from about 100 mg to about 600 mg of said methylsulfonylmethane in one dosage.

7. The supplement composition of claim 1 further comprising an effective amount of S-Adenosylmethionine.

8. The supplement composition of claim 1 further comprising effective amounts of:
silymarin (from milk thistle seed extract);
N-acetyl L-cysteine; and
cruciferious blend comprising broccoli (brassica oleracea var. italica), kale (brassica oleracea var. acephala), and radish (raphanus sativus).

9. The supplement composition of claim 8, wherein said composition comprises from about 75 mg to about 300 mg of said silymarin in one dosage.

10. The supplement composition of claim 8, wherein said composition comprises from about 35 mg to about 150 mg of said N-acetyl L-cysteine in one dosage.

11. The supplement composition of claim 8, wherein said composition comprises from about 75 mg to about 300 mg of said cruciferious blend comprising broccoli (brassica oleracea var. italica), kale (brassica oleracea var. acephala), and radish (raphanus sativus) in one dosage.

12. The supplement composition of claim 8 further comprising an effective amount of S-Adenosylmethionine.

13. A supplement composition for enhancement of methylation process, comprising in one dosage:
(a) from about 75 mg to about 400 mg of vitamin B₆ (as pyridoxine HCl);
(b) from about 35 μg to 200 μg of folic acid;
(c) from about 125 μg to 700 μg of vitamin B₁₂ (as cyanocobalamin);
(d) from about 125 mg to 700 mg of betaine HCl; and
(e) from about 100 mg to 600 mg of methylsulfonylmethane.

14. The supplement composition of claim 13 further comprising from about 200 mg to about 1200 mg of S-adenosylmethionine (as S-adenosylmethionine disulfate ditosylate).

15. The supplement composition of claim 13 further comprising in one dosage:
from about 75 mg to about 300 mg of silymarin (from milk thistle seed extract);
from about 35 mg to about 150 mg of N-acetyl L-cysteine; and
from about 75 mg to about 300 mg of cruciferious blend comprising broccoli (brassica oleracea var. italica), kale (brassica oleracea var. acephala), and radish (raphanus sativus).

16. The supplement composition of claim 15 further comprising from about 200 mg to about 1200 mg of S-adenosylmethionine (as S-adenosylmethionine disulfate ditosylate).

17. A method for enhancement of methylation process comprising orally administering a supplement composition to a person daily in a dosage comprising from about 75 mg to about 400 mg of vitamin B₆ (as pyridoxine HCl), from about 35 μg to 200 μg of folic acid, from about 125 μg to 700 μg of vitamin B₁₂ (as cyanocobalamin), from about 125 mg to 700 mg of betaine HCl, and from about 100 mg to 600 mg of methylsulfonylmethane.

18. The method of claim 17, wherein said supplement composition further comprising in said dosage from about 200 mg to about 1200 mg of S-adenosylmethionine (as S-adenosylmethionine disulfate ditosylate).

19. The method of claim 17, wherein said supplement composition further comprising in said dosage from about 75 mg to about 300 mg of silymarin (from milk thistle seed extract), from about 35 mg to about 150 mg of N-acetyl L-cysteine, and from about 75 mg to about 300 mg of cruciferious blend comprising broccoli (brassica oleracea var. italica), kale (brassica oleracea var. acephala), and radish (raphanus sativus).

20. The method of claim 19, wherein said supplement composition further comprising in said dosage from about 200 mg to about 1200 mg of S-adenosylmethionine (as S-adenosylmethionine disulfate ditosylate).
